(19) United States
(12) Patent Application Publication
(10) Pub. No.: US 2014/0355735 A1
(43) Pub. Date: Dec. 4, 2014

(54) X-RAY IMAGING APPARATUS AND CONTROL METHOD THEREOF

(71) Applicant: SAMSUNG ELECTRONICS CO., LTD., Suwon-si (KR)

(72) Inventors: Ji Young CHOI, Suwon-si (KR); Jong Ha LEE, Hwaseong-si (KR); Young Hun SUNG, Hwaseong-si (KR); Kwang Eun JANG, Seoul (KR)

(73) Assignee: SAMSUNG ELECTRONICS CO., LTD., Suwon-si (KR)

(21) Appl. No.: 14/264,175

(22) Filed: Apr. 29, 2014

(30) Foreign Application Priority Data
May 31, 2013 (KR) 10-2013-0062650

(51) Int. Cl. A61B 6/00; A61B 6/02; A61B 6/03

(52) U.S. Cl. CPC A61B 6/544; A61B 6/032; A61B 6/022
USPC 378/8; 378/41; 378/54

(57) ABSTRACT

Disclosed herein are an X-ray imaging apparatus capable of reducing a dose of radiation, and a control method thereof. The X-ray imaging apparatus includes: a gantry configured to rotate around an object, the object being placed on a table that is configured to be transported into a bore; a depth camera provided on the gantry, the depth camera configured to acquire a depth image of the object; an image processor configured to acquire thickness information of the object from the depth image of the object; and a controller configured to set a dose of X-rays to be irradiated to the object according to the thickness information of the object.

### CROSS-REFERENCE TO RELATED APPLICATION

This application claims the benefit of Korean Patent Application No. 10-2013-0062650, filed on May 31, 2013 in the Korean Intellectual Property Office, the disclosure of which is incorporated herein by reference in its entirety.

### BACKGROUND

1. Field

Exemplary embodiments relate to an X-ray imaging apparatus capable of reducing a dose of radiation, and a control method thereof.

2. Description of the Related Art

An X-ray imaging apparatus is an imaging apparatus configured to irradiate X-rays to an object (e.g., a human body or a product) to visualize the inside of the object. Generally, the X-ray imaging apparatus is used to detect an abnormality such as lesions in human bodies in a medical field or the like, or to understand the inside structures of objects or elements. Also, the X-ray imaging apparatus is used for other purposes, such as, for example, to check baggage in an airport.

Different types of X-ray imaging apparatuses include Digital Radiography (DR), Computed Tomography (CT), and Full Field Digital Mammography (FFDM).

The operation principle of an X-ray imaging apparatus is as follows. An X-ray imaging apparatus irradiates X-rays to an object (e.g., a human body or a product) and then receives X-rays transmitted through (or not transmitted through) the object. Then, the X-ray imaging apparatus converts the received X-rays into electrical signals, and reads out the electrical signals, thereby generating an X-ray image. The X-ray image is displayed by a display so that a user can understand the inside structure of the object.

### SUMMARY

Therefore, it is an aspect of the exemplary embodiments to provide an X-ray imaging apparatus capable of reducing a dose of radiation, and a control method thereof.

Additional aspects of the exemplary embodiments will be set forth in part in the description which follows and, in part, will be obvious from the description, or may be learned by practice of the exemplary embodiments.

In accordance with an aspect of an exemplary embodiment, an X-ray imaging apparatus includes: a gantry configured to rotate around an object, the object being placed on a table that is configured to be transported into a bore; a depth camera provided on the gantry, the depth camera configured to acquire a depth image of the object; an image processor configured to acquire thickness information of the object from the depth image of the object; and a controller configured to set a dose of X-rays to be irradiated to the object according to the thickness information of the object.

In accordance with another aspect of an exemplary embodiment, an X-ray imaging apparatus includes: a gantry configured to rotate around an object, the object being placed on a table that is configured to be transported into a bore; a stereo camera provided on the gantry, the depth camera configured to acquire at least one pair of a left image and a right image of the object; an image processor configured to acquire thickness information of the object from the at least one pair of the left image and the right image of the object; and a controller configured to set a dose of X-rays that is to be irradiated to the object according to the thickness information of the object.

Therefore, according to the X-ray imaging apparatus according to exemplary embodiments, since information regarding the thickness of the object can be acquired without performing a pre-shot, it is possible to reduce a dose of radiation.

### BRIEF DESCRIPTION OF THE DRAWINGS

These and/or other aspects of the exemplary embodiments will become apparent and more readily appreciated from the following description of the exemplary embodiments, taken in conjunction with the accompanying drawings of which:

FIG. 1 is a perspective view of an X-ray imaging apparatus according to an exemplary embodiment;

FIGS. 2A, 2B, and 2C are views for describing a process for acquiring position information and thickness information of an object in the X-ray imaging apparatus;

FIG. 3 is a block diagram illustrating a control configuration of an X-ray imaging apparatus according to an exemplary embodiment;

FIG. 4 illustrates a structure of an X-ray tube included in an X-ray generator according to an exemplary embodiment;

FIG. 5 illustrates a structure of an X-ray detector according to an exemplary embodiment;

FIG. 6 is a view for describing the operation principle of a depth camera illustrated in FIG. 3 according to an exemplary embodiment;

FIG. 7 is a block diagram illustrating a configuration of an image processor illustrated in FIG. 3 according to an exemplary embodiment;

FIG. 8 is a flowchart illustrating a control method of the X-ray imaging apparatus when thickness information of an object is acquired after the depth camera is fixed according to an exemplary embodiment;

FIG. 9 is a flowchart illustrating a control method of the X-ray imaging apparatus when thickness information of an object is acquired at different positions of the depth camera moving around the object according to an exemplary embodiment;

FIG. 10 is a block diagram illustrating a control configuration of an X-ray imaging apparatus according to another exemplary embodiment;

FIG. 11 is a block diagram illustrating a configuration of an image processor illustrated in FIG. 10;

FIG. 12 is a flowchart illustrating a control method of the X-ray imaging apparatus when thickness information of an object is acquired after a stereo camera is fixed according to an exemplary embodiment; and

FIG. 13 is a flowchart illustrating a control method of the X-ray imaging apparatus when thickness information of an object is acquired at different positions of the stereo camera moving around the object.

### DETAILED DESCRIPTION

Hereinafter, exemplary embodiments of an X-ray imaging apparatus and a control method thereof will be described with reference to the accompanying drawings, wherein like reference numerals refer to like elements throughout.

Different types of X-ray imaging apparatuses include Digital Radiography (DR), Computed Tomography (CT), and Full Field Digital Mammography (FFDM). In the following description, the X-ray imaging apparatus is assumed to be CT, but it is understood that the X-ray imaging apparatuses according to other exemplary embodiments are not limited to being CT.

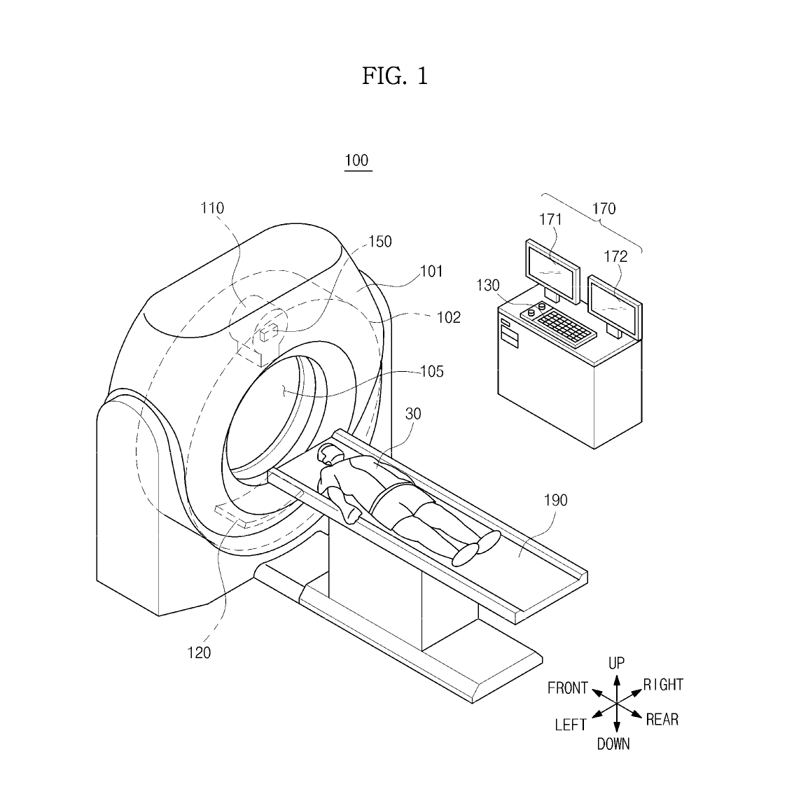

FIG. 1 is a perspective view of an X-ray imaging apparatus according to an exemplary embodiment.

Referring to FIG. 1, an X-ray imaging apparatus 100 may include a housing 101, a table 190, an input unit 130, and a display unit 170.

A gantry 102 is installed in the housing 101. In the gantry 102, an X-ray generator 110 and an X-ray detector 120 are disposed to be opposite to each other. The gantry 102 rotates at an angle ranging from 180° to 360° around a bore 105. When the gantry 102 rotates, the X-ray generator 110 and the X-ray detector 120 rotate accordingly.

A depth camera 150 is provided near the X-ray generator 110. The depth camera 150 is used to photograph an object 30 and acquire a depth image of the object 30. The depth camera 150 may be disposed on the gentry 102, and accordingly, the depth camera 150 rotates together with the gantry 102 when the gantry 102 rotates.

The table 190 transports the object 30 that is to be photographed into the bore 105. The table 190 may move in front-rear, up-down, left-right, and up-down directions while maintaining horizontality with respect to a ground.

The input unit 130 receives instructions or commands for controlling operations of the X-ray imaging apparatus 100. To receive the instructions or commands, the input unit 130 may include at least one of a keyboard and a mouse.

The display unit 170 displays an X-ray image of the object 30. The X-ray image may be a 2-Dimensional (2D) projected image, a 3D image, or a 3D stereo image of the object 30.

According to an exemplary embodiment, the 2D projected image of the object 30 is acquired by detecting X-rays transmitted through the object 30 after irradiating X-rays to the object. The 3D image of the object 30 is acquired by performing volume rendering on 3D volume data restored from a plurality of 2D projected images with respect to a predetermined viewpoint. That is, a 3D image is a 2D reprojected image acquired by reprojecting volume data onto a 2D plane (that is, a display screen) with respect to a predetermined viewpoint. Meanwhile, the 3D stereo image of the object 30 is acquired by performing volume rendering on volume data with respect to left and right viewpoints corresponding to a human's left and right eyes to acquire a left image and a right image, and synthesizing the left image with the right image.

The display unit 170 includes at least one display. FIG. 1 shows a case in which the display unit 170 includes a first display 171 and a second display 172. In this case, the first display 171 and the second display 172 may display different types of images. For example, the first display 171 may display a 2D projected image, and the second display 172 may display a 3D image or a 3D stereo image. Alternatively, the first and second displays 171 and 172 may display the same type of images.

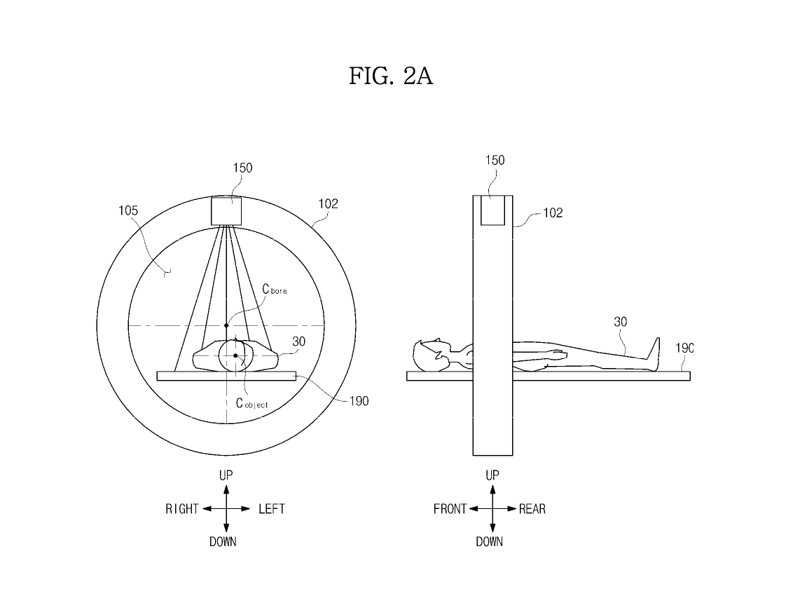

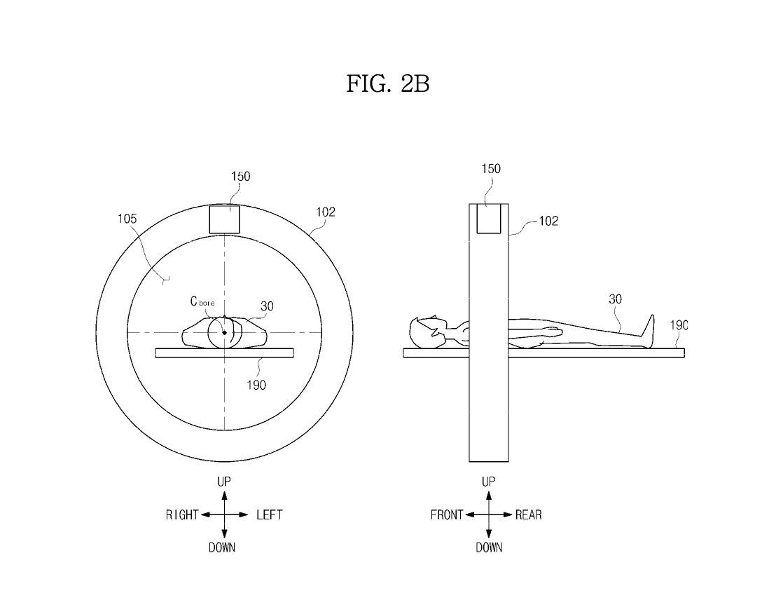

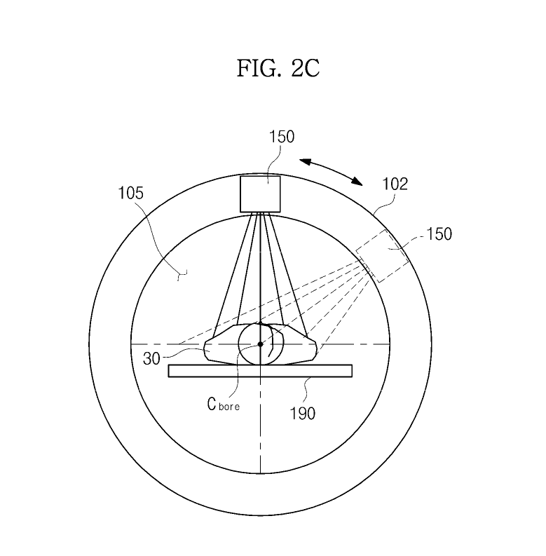

The external appearance of the X-ray imaging apparatus 100 according to an exemplary embodiment has been described. The X-ray imaging apparatus 100 may acquire at least one piece of position information and thickness information of the object 30 using the depth camera 150. This operation will be described in detail with reference to FIGS. 2A, 2B, and 2C, below. In FIGS. 2A, 2B, and 2C, the left drawings are rear views of the X-ray imaging apparatus 100, and the right drawings are side views of the X-ray imaging apparatus 100.

As illustrated in FIG. 2A, after the table 190 is transported into the bore 105, the depth camera 150, which faces the table 190, photographs the object 30. Then, a depth image of the object 30 is acquired by the depth camera 150, and the depth image of the object 30 is analyzed to acquire position information and thickness information of the object 30, where the thickness information of the object 30 may represent a length from the table 190 to the topmost point of the object 30, the position information of the object 30 may represent a location of a center C_{object} of the object 30, and the center C_{object} of the object 30 may represent an intersection of the thickness of the object 30 and the width of the object 30. For example, as illustrated in FIG. 2A, when the chest of the human body is photographed by the X-ray imaging apparatus 100, a center C_{object} of the chest may be an intersection of the chest's thickness and the chest's width.

The position information of the object 30 may be used to adjust the position of the table 190. More specifically, the center C_{object} of the object 30 is compared to a center C_{bore} of the bore 105, and a direction in which the table 190 should be moved and a distance by which the table 190 should be moved in order to position the center C_{object} of the object 30 in an identical position as the center C_{bore} of the bore 105 are determined and calculated based on the results of the comparison. Then, the table 190 is moved by the calculated distance in the determined direction. For example, as illustrated in FIG. 2A, if the center C_{object} of the object 30 is positioned to the left and down from the center C_{bore} of the bore 105, the table 190 is moved to the right and up by a distance by which the center C_{object} of the object 30 deviates from the center C_{bore} of the bore 105 so that a position of the center C_{object} of the object 30 becomes identical to a position of the center C_{bore} of the bore 105. As such, by making the position of the center C_{object} of the object 30 identical to the position of the center C_{bore} of the bore 105, a clearer 3D image may be restored from at least one X-ray image.

After the position of the table 190 is adjusted, a dose (e.g., quantity) of X-rays that is to be irradiated to the object 30 may be set according to the thickness information of the object 30. Then, the X-ray generator 110 may irradiate the set dose of X-rays to the object 30. As such, by setting a dose of X-rays according to thickness information of the object, a pre-shot which irradiates a low dose of X-rays to the object to check transparency of X-rays with respect to the object does not need to be performed. Accordingly, it is possible to reduce a dose of radiation that is applied to an object.

As another example, after the position of the table 190 is adjusted, as illustrated in FIG. 2C, the gantry 102 may rotate to move the depth camera 150 around the object 30. Then, the depth camera 150 photographs the object 30 at different positions while moving around the object 30 together with the gantry 102, acquires a plurality of depth images of the object 30, and acquires a plurality of pieces of thickness information of the object 30 from the depth images of the object 30. Thereafter, a dose of X-rays is set for each piece of the thickness information of the object 30. That is, a dose of X-rays is set for each position of the depth camera 150 at which thickness information has been acquired. Thereafter, when the gentry 102 rotates such that the X-ray generator 110 arrives at a position at which thickness information has already been acquired, the X-ray generator 110 irradiates a dose of X-rays corresponding to the position to the object 30.

In this way, by setting a dose of X-rays according to thickness information of the object, a clearer X-ray image may be acquired at each position. Referring to the example of FIG. 2C, when the depth camera 150 is provided on the X-ray generator 110, the thickness of the object 30, that is, a length by which X-rays pass through the object 30, varies as the X-ray generator 110 moves around the object 30. If the same dose of X-rays is irradiated to the object 30 without considering the thickness of the object 110 according to the position of the X-ray generator 110, acquired X-ray images may have different qualities. However, if an appropriate dose of X-rays is set for each position of the X-ray generator 110 in consideration of the thickness of the object 30 measured at the position of the X-ray generator 110, and the set dose of X-rays is irradiated to the object 30 at the position of the X-ray generator, X-ray images having a uniform quality can be obtained.

FIG. 2C shows a case of moving the depth camera 150 after adjusting the position of the table 190, and then acquiring thickness information of the object 30 at each position of the depth camera 150. However, an operation of adjusting the position of the table 190 is not necessarily performed prior to an operation of moving the depth camera 150. More specifically, after the depth camera 150 moves in the state as illustrated in FIG. 2A to acquire a depth image of the object 30 at each position of the depth camera 150 and acquire position information and thickness information of the object 30 from the depth image of the object 30, the position of the table 190 may be adjusted according to the position information of the object 30, and a dose of X-rays may be set based on thickness information of the object 30 according to each position of the depth camera 150.

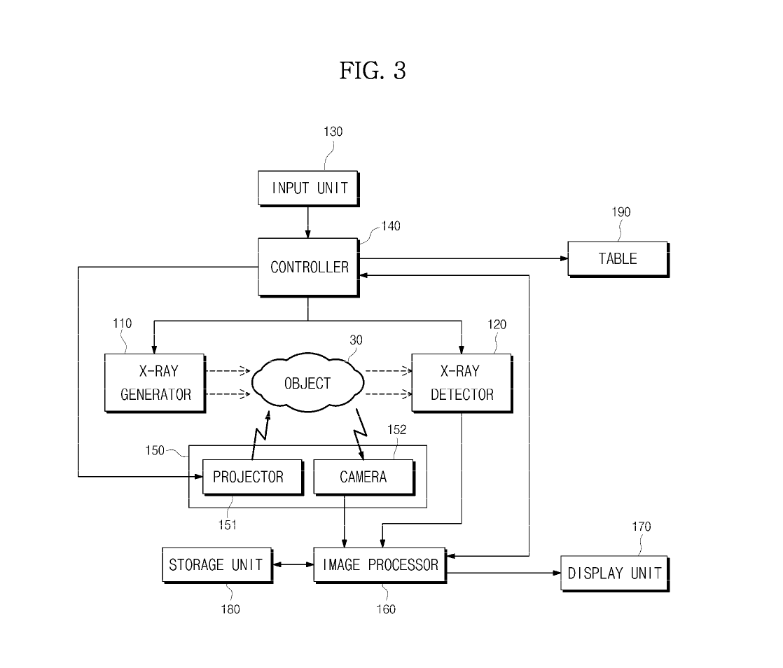

FIG. 3 is a block diagram illustrating a control configuration of the X-ray imaging apparatus 100 according to an exemplary embodiment.

Referring to FIG. 3, the X-ray imaging apparatus 100 includes the X-ray generator 110, the X-ray detector 120, the input unit 130, a controller 140, the depth camera 150, an image processor 160, the display unit 170, a storage unit 180, and the table 190.

The input unit 130 receives, as described above, instructions or commands for controlling operations of the X-ray imaging apparatus 100.

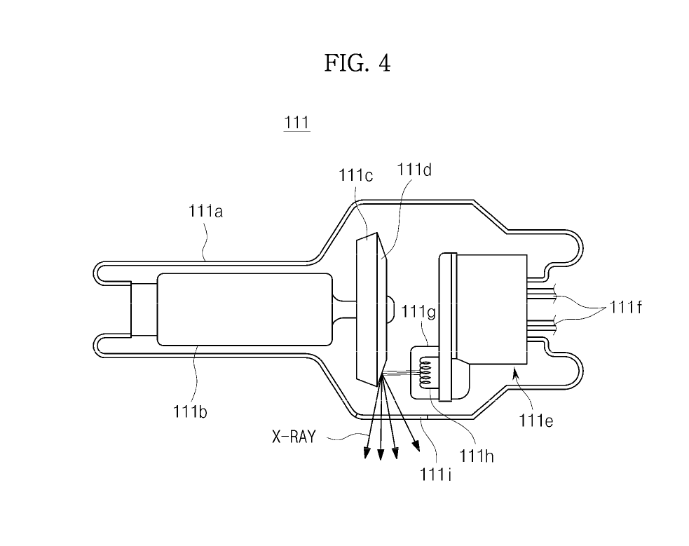

The X-ray generator 110 generates X-rays, and irradiates the X-rays to an object 30. The X-ray generator 110 includes an X-ray tube for generating X-rays. The X-ray tube will be described in detail with reference to FIG. 4, below.

FIG. 4 illustrates a structure of an X-ray tube 111 included in the X-ray generator 110 according to an exemplary embodiment.

Referring to FIG. 4, the X-ray tube 111 may be implemented as a two-electrode vacuum tube including an anode 111c and a cathode 111e. The body of the two-electrode vacuum tube 111 may be a glass bulb 111a made of silica (hard) glass or the like.

The cathode 111e includes a filament 111h and a focusing electrode 111g for focusing electrons. The focusing electrode 111g is also referred to as a focusing cup. When the inside of the glass bulb 111a is kept in a high vacuum environment of about 10 mmHg, and the filament 111h of the cathode 111e is heated to a high temperature, thermoelectrons are generated. The filament 111h may be a tungsten filament, and the filament 111h may be heated when current is applied to electric leads connected to the filament 111h. However, implementing the filament 111h in the cathode 111e is only exemplary, and it is also possible to use a carbon nano-tube capable of being driven with high-speed pulses, as a cathode.

The anode 111c is primarily made of copper, and a target material 111d is disposed or applied on one side of the anode 111c facing the cathode 111e. The target material 111d may be a high-Z material, e.g., Cr, Fe, Co, Ni, W, and Mo. As the melting point of the target material 111d increases, focal spot size may decrease.

When a high voltage is applied between the cathode 111e and the anode 111c, thermoelectrons are accelerated and collide with the target material 111d of the anode 111c, thereby generating X-rays. The X-rays are irradiated to the outside through a window 111i. The window 111i may be a Beryllium (Be) thin film. Also, a filter (not shown) for filtering a specific energy band of X-rays may be provided on the front or rear side of the window 111i.

The target material 111d may be rotated by a rotor 111b. When the target material 111d rotates, the heat accumulation rate may increase 10 times per unit area and the focal spot size may be reduced, compared to when the target material 111d is fixed.

The voltage that is applied between the cathode 111e and the anode 111c of the X-ray tube 111 is called a tube voltage. The magnitude of a tube voltage may be expressed as a crest value (kVp).

When the tube voltage increases, a velocity of thermoelectrons increases accordingly. Then, energy (energy of photons) of X-rays that are generated when the thermoelectrons collide with the target material 111d also increases. As the energy of the X-rays increases, a larger amount of X-rays are transmitted through the object 30. Accordingly, the X-ray detector 120 (see FIG. 3) also will detect a large amount of X-rays. As a result, an X-ray image having a high Signal-to-Noise Ratio (SNR), that is, an X-ray image having high quality, can be obtained.

On the contrary, when the tube voltage decreases, a velocity of thermoelectrons decreases accordingly. Then, energy (energy of photons) of X-rays that are generated when the thermoelectrons collide with the target material 111d also decreases. As the energy of the X-rays decreases, a larger amount of X-rays are absorbed in the target 30. Accordingly, the X-ray detector 120 will detect a small amount of X-rays. As a result, an X-ray image having a low SNR, that is, an X-ray image having low quality, will be obtained.

Current flowing through the X-ray tube 111 is called tube current, and can be expressed as an average value (mA). When tube current increases, a dose of X-rays (that is, X-ray photons) increases so that an X-ray image having a high SNR is obtained. On the contrary, when tube current decreases, a dose of X-rays decreases so that an X-ray image having a low SNR is obtained.

In summary, the energy of X-rays can be controlled by adjusting a tube voltage. Also, a dose or intensity of X-rays can be controlled by adjusting tube current and an X-ray exposure time. In other words, by controlling a tube voltage or tube current according to the kind or properties of an object, an energy or dose of X-rays to be irradiated can be controlled.

X-rays that are irradiated from the X-ray generator 110 (see FIG. 3) have a specific energy band that is defined by upper and lower limits. The upper limit of the specific energy band, that is, a maximum energy of X-rays to be irradiated, may be adjusted according to the magnitude of a tube voltage. The lower limit of the specific energy band, that is, a minimum energy of X-rays to be irradiated, may be adjusted by a filter included in the X-ray generator 110. More specifically, by filtering out X-rays having a low energy band using the filter, an average energy of X-rays to be irradiated can be increased. The energy of X-rays to be irradiated may be expressed as a maximum energy or an average energy.

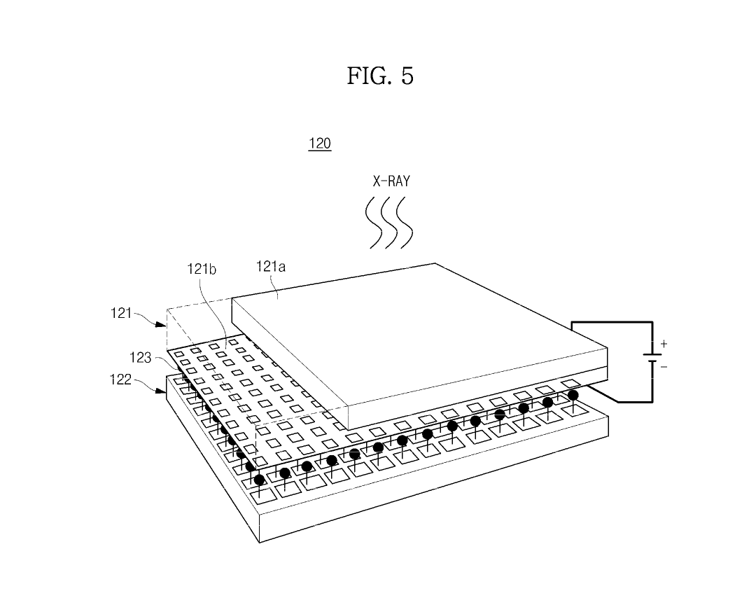

Referring again to FIG. 3, the X-ray detector 120 detects X-rays transmitted through the object 30, and converts the X-rays into electrical signals. The X-ray detector 120 will be described in more detail with reference to FIG. 5, below.

FIG. 5 illustrates a structure of the X-ray detector 120 according to an exemplary embodiment.

Referring to FIG. 5, the X-ray detector 120 includes a light receiving device 121 to detect X-rays and convert the X-rays into electrical signals, and a read circuit 122 to read out the electrical signals. According to an exemplary embodiment, the read circuit 122 is implemented in the form of a 2D pixel array including a plurality of pixel areas. The light receiving device 121 may be made of a single crystal semiconductor material in order to ensure high resolution, high response speed, and a high dynamic area even under conditions of low energy and a small dose of X-rays. The single crystal semiconductor material may be Ge, CdTe, CdZnTe, or GaAs, although is not limited thereto and may also be implemented as other materials.

The light receiving device 121 may be implemented in the form of a PIN photodiode. The PIN photodiode is fabricated by bonding a p-type layer 121b in which p-type semiconductors are arranged in the form of a 2D pixel array on the lower surface of an n-type semiconductor substrate 121a having a high resistance. The read circuit 122, which is fabricated according to a Complementary Metal Oxide Semiconductor (CMOS) process, is coupled with the light receiving device 121 in units of pixels. The CMOS read circuit 122 and the light receiving device 121 may be coupled by a Flip-Chip Bonding (FCB) method. More specifically, the CMOS read circuit 122 and the light receiving device 121 may be coupled by forming bumps 123 with PbSn, In, or the like, reflowing, applying heat, and then compressing. However, the X-ray detector 120 is not limited to this structure and may be implemented as various other structures according to other exemplary embodiments.

Referring again to FIG. 3, the depth camera 150 photographs the object 30 lying on the table 190 to acquire a depth image of the object 30.

For example, the depth camera 150 may be implemented as a structured-light type depth camera. The structured-light type depth camera 150 projects a specific pattern of structured light to an object, and photographs a light pattern distorted by the object, thereby acquiring a depth image of the object.

As another example, the depth camera 150 may be implemented as a Time Of Flight (TOF) type depth camera. The TOF type depth camera 150 irradiates a predetermined signal to an object, measures a time taken by a signal reflected from the object to arrive at the depth camera 150, and acquires a depth image of the object based on the measured time. The predetermined signal irradiated from the TOF type depth camera 150 to the object may be infrared light or an ultrasonic signal. In the following description, for convenience of description, the depth camera 150 is assumed to be a structured-light type depth camera.

As illustrated in FIG. 3, the structured-light type depth camera 150 may include a projector 151 and a camera 152. The projector 151 projects structured light to the object 30. The structured light is light having a specific pattern. The camera 152 photographs a light pattern distorted by the object 30. The camera 152 may include an image sensor. The image sensor may be implemented as a Charge Coupled Device (CCD) image sensor or a CMOS image sensor.

The structured-light type depth camera 150 may use an optical spot method, an optical slit method, or an optical grid method, according to the kind of structured light that is irradiated from the projector 151 to the object 30.

The optical spot method is a technique of projecting an optical spot having a color that can be easily identified when the optical spot is shown on the surface of an object, to the surface of the object. According to the optical spot method, by moving an optical spot along the ridge of an object, the shape of the object can be recognized.

The optical slit method is a technique of projecting a slit image of light to the surface of an object. When a slit pattern is projected to the surface of the object, the slit pattern appears as a long line on the surface of the object. Accordingly, when the object has been photographed by the camera 152, the slit image on the object can be easily recognized. The optical slit method is also called an optical cut method since an object is shown as if the object is cut by an optical slit.

The optical grid method is a technique of projecting an optical lattice image to the surface of an object. According to the optical grid method, an image of a plurality of lattices is shown on the surface of an object. Accordingly, the optical grid method is used to measure a 3D position of an object using a plurality of points irradiated on the object.

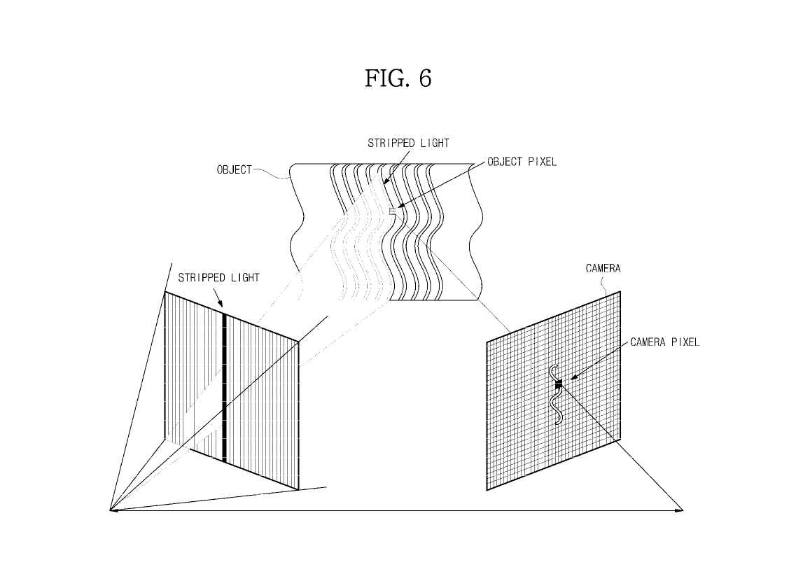

FIG. 6 is a view for describing the operation principle of the structured-light type depth camera 150 illustrated in FIG. 3 according to an exemplary embodiment. FIG. 6 shows a case in which the projector 151 of the depth camera 150 projects a stripped pattern of light to the object 30. If a stripped pattern of light is projected to the object 30, the stripped pattern of light is distorted by the curved surface of the object 30. Then, the distorted pattern of light appearing on the surface of the object 30 is photographed, and the distorted pattern of light is compared to the stripped pattern of light projected to the object 30 so that 3D information (that is, a depth image) about the object 30 is obtained.

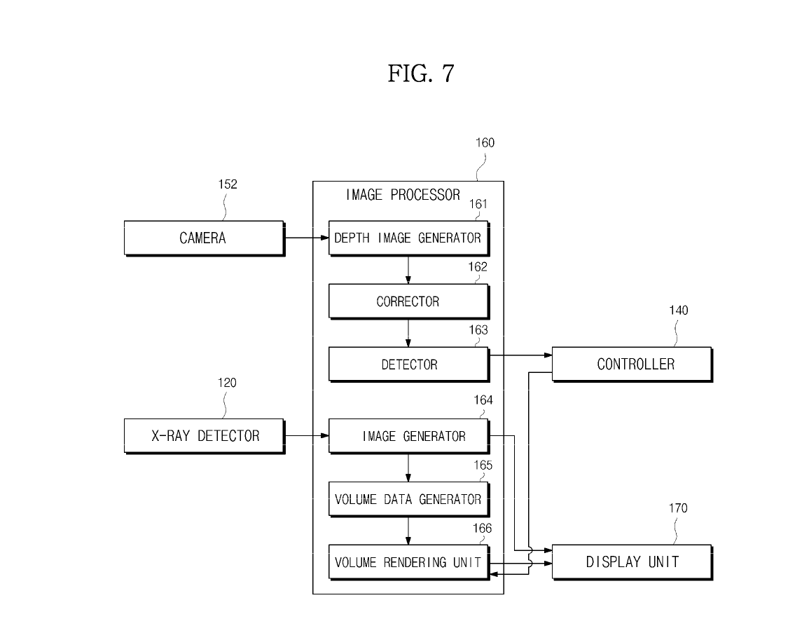

Referring again to FIG. 3, the image processor 160 may generate a depth image of the object 30 based on electrical signals output from the individual pixels of the camera 152, and acquire position information and thickness information of the object 30 from the depth image of the object 30. Also, the image processor 160 may generate an X-ray image based on electrical signals output from the individual pixels of the X-ray detector 120. The image processor 160 will be described in more detail with reference to FIG. 7, below.

Referring to FIG. 7, the image processor 160 includes a depth image generator 161, a corrector 162, a detector 163, an image generator 164, a volume data generator 165, and a volume rendering unit 166.

The depth image generator 161 generates a depth image of the object 30 based on electrical signals output from the individual pixels of the camera 152. The depth image of the object 30 may be provided to the corrector 162.

The corrector 162 corrects the depth image of the object 30. For example, if the table 190 which should be a flat plane is shown as an image of a curved plane, the corrector 162 may correct the image of the table 190 to an image of a flat plane. As another example, when light reflected from the surface of the table 190 is scattered by strong ambient lighting so that incorrect depth information of the table 190 is acquired, the corrector 172 may correct the incorrect depth information. A corrected depth image may be provided to the detector 163.

The detector 163 acquires position information and thickness information of the object 30 from the corrected depth image. The position information and thickness information of the object 30 may be provided to the controller 140 which will be described later. The position information of the object 30 may be used to adjust the position of the table 190, and the thickness information of the object 30 may be used to set a dose of X-rays that is to be irradiated to the object 30.

The image generator 164 generates a plurality of 2D projected images based on electrical signals output from the individual pixels of the X-ray detector 120. As described above, the X-ray generator 110 and the X-ray detector 120 rotate at a predetermined angle around the object 30 when the gantry 102 rotates, so that a plurality of 2D projected images of the object 30 are acquired to correspond to different positions of the depth camera 150.

The volume data generator 165 reconstructs the 2D projected images acquired at the different positions to generate 3D volume data about the object 30. Reconstructing 2D projected images refers to a process of reconstructing an object represented in two dimensions in a 2D projected image to a 3D image that looks similar to a real object. Methods of reconstructing 2D projected images include, for example, an iterative method, a non-iterative method, a Direct Fourier (DF) method, and a back projection method.

The iterative method is a method of continuously correcting projection data until data representing a structure similar to the original structure of an object is obtained. The non-iterative method is a method of applying an inverse-transform function of a transform function used to model a 3D object to a 2D image to a plurality of pieces of projection data to reconstruct 2D images to a 3D image. An example of the non-iterative method is Filtered Back-Projection (FBP). The FBP technique is a method of filtering projection data to cancel blurs formed around the center portion of a projected image and then back-projecting. The DF method is a method of transforming projection data from a spatial domain to a frequency domain. The back projection method is a method of reconstructing projection data acquired at a plurality of viewpoints on a screen.

The volume data generator 165 generates 3D volume data about the object 30 from a plurality of 2D projected images using one of the above-described methods. Instead of acquiring a plurality of 2D projected images by rotating the X-ray generator 110 and the X-ray detector 120 with respect to the object 30, when a plurality of section images about the object 30 are acquired by moving the X-ray generator 110 and the X-ray detector 120 using a different method, 3D volume data of the object 30 may be generated by accumulating the plurality of section images of the object 30 in a vertical-axis direction.

The volume data may be represented as a plurality of voxels. The term “voxel” is formed from the words “volume” and “pixel”. If a pixel is defined as a point on a 2D plane, a voxel is defined as a point in a 3D space. Accordingly, a pixel includes X and Y coordinates, and a voxel includes X, Y, and Z coordinates.

The volume rendering unit 166 performs volume rendering on the 3D volume data to generate a 3D image and a 3D stereoscopic image. The volume rendering can be classified into surface rendering and direct volume rendering.

Surface rendering is a technique which includes extracting surface information from volume data based on predetermined scalar values and amounts of spatial changes, converting the surface information into a geometric factor, such as a polygon or a curved patch, and then applying a conventional rendering technique to the geometric factor. Examples of the surface rendering technique include a marching cubes algorithm and a dividing cubes algorithm.

The direct volume rendering technique includes directly rendering volume data without converting volume data into a geometric factor. The direct volume rendering technique is useful to represent a translucent structure since the direct volume rendering technique can visualize the inside of an object. The direct volume rendering technique may be classified into an object-order method and an image-order method according to a way of approaching volume data.

The object-order method includes searching for volume data in its storage order and synthesizing each voxel with the corresponding pixel. A representative example of the object-order method is splatting.

The image-order method includes sequentially deciding pixel values in the order of scan lines of an image. Examples of the image-order method include Ray-Casting and Ray-Tracing.

The Ray-Casting technique includes irradiating a virtual ray from a specific viewpoint toward a predetermined pixel of a display screen, and detecting voxels through which the virtual ray has been transmitted from among voxels of volume data. Then, brightness values of the detected voxels are accumulated to decide a brightness value of the corresponding pixel of the display screen. Alternatively, an average value of the detected voxels may be decided as a brightness value of the corresponding pixel of the display screen. Also, a weighted average value of the detected voxels may be decided as a brightness value of the corresponding pixel of the display screen.

The Ray-Tracing technique includes tracing a path of a ray coming to an observer's eyes. Unlike Ray-Casting which includes detecting an intersection at which a ray meets volume data, Ray-Tracing can trace an irradiated ray and thereby reflect how the ray travels, such as reflection, refraction, etc. of the ray.

The Ray-Tracing technique can be classified into Forward Ray-Tracing and Backward Ray-Tracing. Forward Ray-Tracing includes modeling a phenomenon in which a ray irradiated from a virtual light source arrives at volume data to be reflected, scattered, or transmitted, thereby finding a ray finally coming to an observer's eyes. Backward Ray-Tracing includes backwardly tracing a path of a ray coming to an observer's eyes.

The volume rendering unit 166 performs volume rendering on 3D volume data using one of the above-described volume rendering methods to generate a 3D image or a 3D stereoscopic image. As described above, according to an exemplary embodiment, a 3D image is a 2D reprojected image acquired by reprojecting volume data to a 2D display screen with respect to a predetermined viewpoint. According to an exemplary embodiment, a 3D stereo image is acquired by performing volume rendering on volume data with respect to two viewpoints corresponding to a human's left and right eyes to acquire a left image and a right image, and synthesizing the left image with the right image.

Referring again to FIG. 3, the storage unit 180 may store data and algorithms required for operations of the image processor 160, and may also store images generated by the image processor 160. The storage unit 180 may be implemented as a volatile memory device, a non-volatile memory device, a hard disk, an optical disk, or a combination thereof. However, the storage unit 180 is not limited to the above-mentioned devices, and may be implemented as any storage device well-known in the art.

The display unit 170 displays images generated by the image processor 160. The display unit 170 includes the first display 171 and the second display 172 as described above.

The controller 140 determines a direction in which the table 190 should be moved and calculates a distance by which the table 190 should be moved, based on the position information of the object 30 received from the detector 163 of the image processor 160, and generates a control signal for moving the table 190 by the calculated distance in the determined direction. The control signal is provided to a driver (not shown) included in the table 190 so as to move the table 190.

Also, the controller 140 sets a dose of X-rays that is to be irradiated to the object 30 according to the thickness information of the object 30 received from the detector 163 of the image processor 160.

If the thickness information of the object 30 is thickness information from a depth image acquired when the depth camera 150 has been fixed, the controller 140 may control the X-ray generator 110 to irradiate a set dose of X-rays regardless of the position of the X-ray generator 110, even though the X-ray generator 110 and the X-ray detector 120 move due to rotation of the gantry 102.

In contrast, if the thickness information of the object 30 is thickness information from a plurality of depth images acquired at different positions of the depth camera 150 moving around the object 30, the controller 140 may set a dose of X-rays for each position of the depth camera 150 based on thickness information acquired at the position of the depth camera 150. Thereafter, when the gantry 102 rotates and thus the X-ray generator 110 arrives at a predetermined position, the controller 140 may control the X-ray generator 110 to irradiate a dose of X-rays corresponding to the predetermined position.

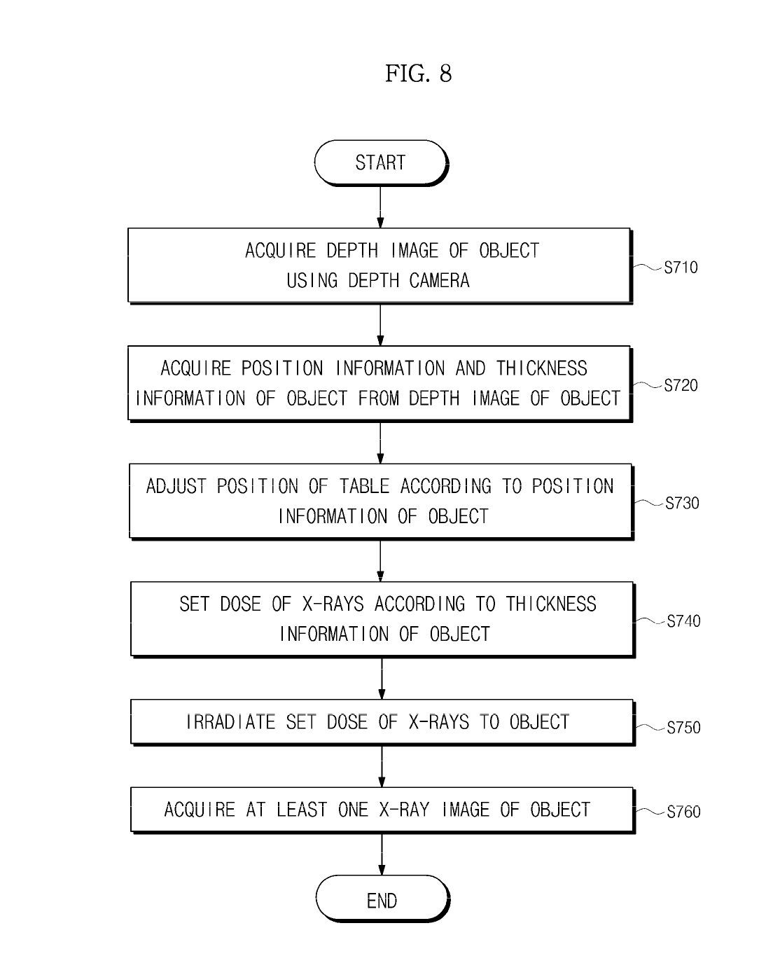

FIG. 8 is a flowchart illustrating a control method of the X-ray imaging apparatus 100 when thickness information of an object is acquired after the depth camera 150 is fixed according to an exemplary embodiment.

Referring to FIGS. 1, 3, and 8, first, the table 190 moves a distance to transport the object 30 into the bore 105. Then, the depth camera 150 moves to face the table 190. In this state, the depth camera 150 photographs the object 30 to acquire a depth image of the object 30 at operation S710. More specifically, the projector 151 of the depth camera 150 projects a predetermined pattern of structured light to the object 30, and the camera 152 of the depth camera 150 photographs the object 30 on which the structured light has been projected. Then, a depth image of the object 30 is acquired based on electrical signals output from the individual pixels of the camera 152.

Successively, position information and thickness information of the object 30 are acquired from the depth image of the object 30 at operation S720. An operation of correcting distortion of the depth image may be selectively performed before acquiring the position information and thickness information of the object 30 from the depth image of the object 30. A determination as to whether to correct the depth image of the object 30 may depend on an instruction or command that is input through the input unit 130, or a user's pre-setting.

Thereafter, the position of the table 190 is adjusted according to the position information of the object 30 at operation S730. More specifically, a direction in which the table 190 should be moved and a distance by which the table 190 should be moved in order to make a position of the center C_{object} of the object 30 identical to a position of the center C_{bore} of the bore 105 are determined and calculated. Then, the table 190 is moved by the calculated distance in the determined direction.

Then, a dose of X-rays that is to be irradiated to the object 30 is set based on the thickness information of the object 30 at operation S740. For example, a tube voltage may be set in proportion to the thickness of the object 30. As another example, tube current may be set in proportion to the thickness of the object 30. As still another example, it is possible to increase both a tube voltage and tube current in proportion to the thickness of the object 30.

Thereafter, when the gantry 102 rotates such that the X-ray generator 110 and the X-ray detector 120 rotate around the object 30, the X-ray generator 110 irradiates the set dose of X-rays to the object 30 at operation S750. At this time, the X-ray generator 110 irradiates the set dose of X-rays to the object 30 regardless of where the X-ray generator 110 is positioned.

By irradiating the set dose of X-rays to the object 30, at least one X-ray image of the object 30 can be acquired at operation S760. According to exemplary embodiments, the X-ray image may be one of: a plurality of 2D projected images of the object 30; a 3D image acquired by performing volume rendering on 3D volume data generated based on the plurality of 2D projected images, with respect to a predetermined viewpoint; and a 3D stereoscopic image acquired by performing volume rendering on the 3D volume data with respect to two viewpoints corresponding to different positions to acquire a left image and a right image, and then synthesizing the left image with the right image. The X-ray image may be displayed by the display unit 170 according to a predetermined display method.

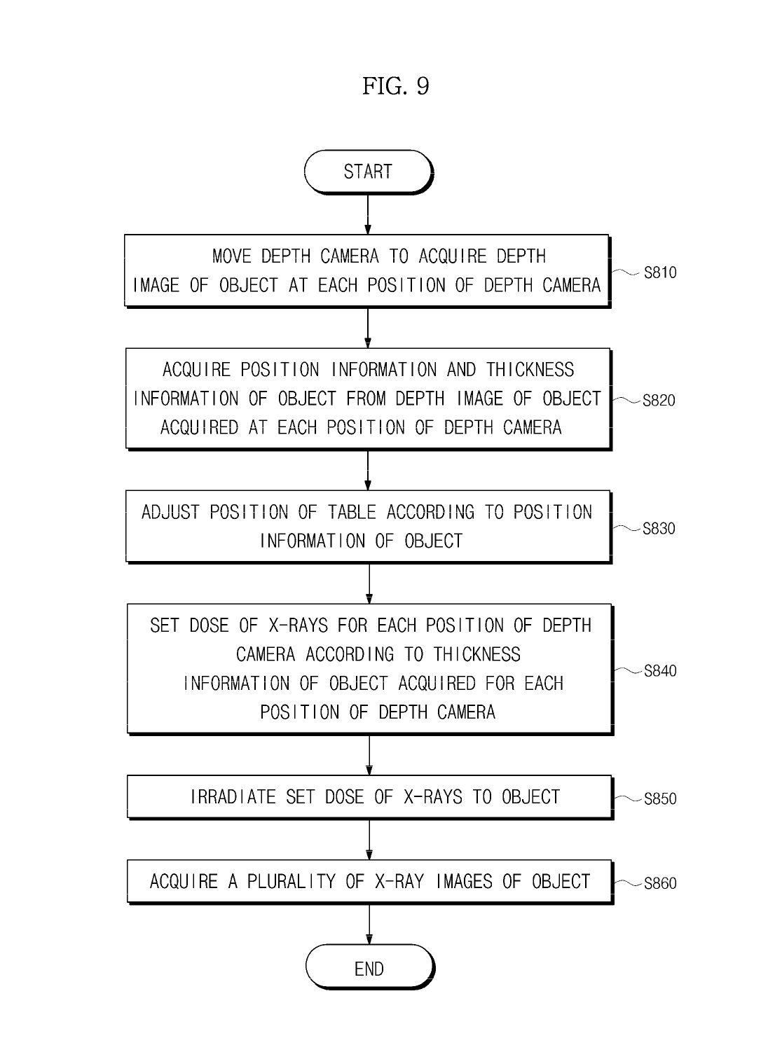

FIG. 9 is a flowchart illustrating a control method of the X-ray imaging apparatus when thickness information of the object 30 is acquired at different positions of the depth camera 150 moving around the object 30.

Referring to FIGS. 1, 3, and 9, first, the table 190 moves a distance to transport the object 30 into the bore 105. Then, the depth camera 150 moves to face the table 190. In this state, the depth camera 150 moves, and a depth image of the object 30 is acquired at each position of the depth camera 150 at operation S810. More specifically, the projector 151 of the depth camera 150 projects a predetermined pattern of structured light to the object 30, and the camera 152 of the depth camera 150 photographs the object 30 on which the structured light has been projected. Then, a depth image of the object is acquired based on electrical signals output from the individual pixels of the camera 152. Operation of projecting the predetermined pattern of structured light and operation of photographing the object 30 on which the structured light has been projected may be sequentially performed. The depth image generator 161 of the image processor 160 may read out electrical signals from the individual pixels of the camera 152 whenever the depth camera 150 arrives at one of predetermined positions, and acquire a depth image of the object 30 for the corresponding position.

Then, position information and thickness information of the object 30 are acquired from a depth image of the object 30 for each position at operation S820. An operation of correcting distortion of each depth image may be selectively performed before acquiring the position information and thickness information of the object 30 from the depth image of the object 30. A determination as to whether to correct each depth image may depend on an instruction or command that is input through the input unit 130, or a user's pre-setting.

Thereafter, the position of the table 190 is adjusted according to the position information of the object 30 at operation S830. More specifically, a direction in which the table 190 should be moved and a distance by which the table 190 should be moved in order to make a position of the center C_{object} of the object 30 identical to a position of the center C_{bore} of the bore 105 are determined and calculated. Then, the table 190 is moved by the calculated distance in the determined direction. At this time, the position information of the object 30 may be selected from among a plurality of pieces of position information acquired from the depth images of the object 30. Alternatively, the position information of the object 30 may be an average value of a plurality of pieces of position information acquired from the depth images of the object 30.

Then, a dose of X-rays that is to be irradiated to the object 30 for each position of the depth camera 150 is set based on thickness information of the object 30 acquired for the position of the depth camera 150 at operation S840. For example, a tube voltage may be set in proportion to the thickness of the object 30. As another example, tube current may be set in proportion to the thickness of the object 30. As still another example, it is possible to increase both a tube voltage and tube current in proportion to the thickness of the object 30.

Thereafter, when the gantry 102 rotates such that the X-ray generator 110 rotates around the object 30, the X-ray generator 110 irradiates a dose of X-rays set for the corresponding position to the object 30. More specifically, whenever the X-ray generator 110 arrives at a position at which thickness information of the object 30 has been acquired, the X-ray generator 110 irradiates a dose of X-rays corresponding to the position to the object 30 at operation S850.

By irradiating a dose of X-rays corresponding to each position to the object 30, a plurality of X-ray images of the object 30 can be acquired at operation S860. According to exemplary embodiments, the X-ray image may be one of: a plurality of 2D projected images of the object 30; a 3D image acquired by performing volume rendering on 3D volume data generated based on the plurality of 2D projected images, with respect to a predetermined viewpoint; and a 3D stereoscopic image acquired by performing volume rendering on the 3D volume data with respect to two viewpoints corresponding to different positions to acquire a left image and a right image, and then synthesizing the left image with the right image. The X-ray image may be displayed through the display unit 170 according to a predetermined display method.

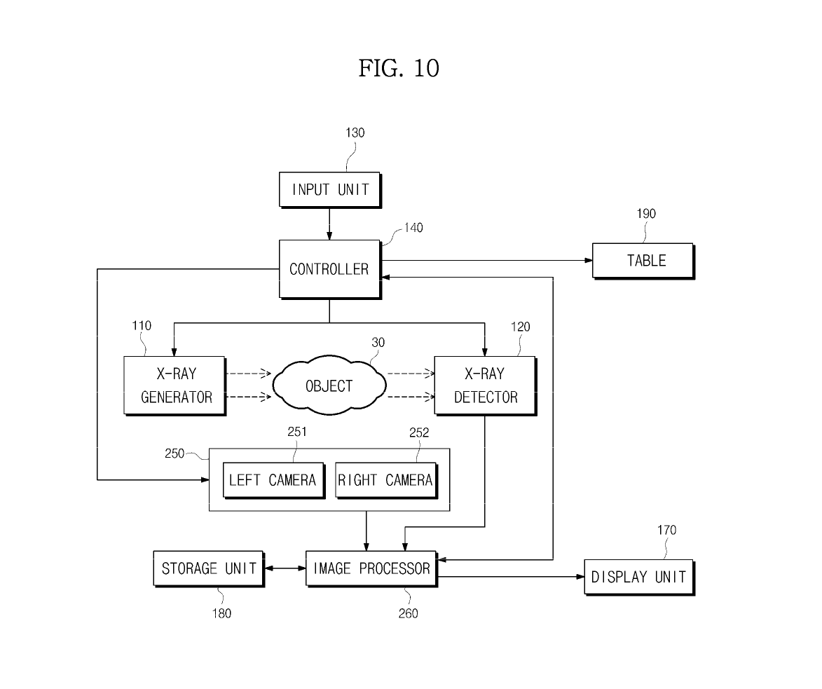

FIG. 10 is a block diagram illustrating a control configuration of an X-ray imaging apparatus 100 according to another exemplary embodiment.

Referring to FIG. 10, the X-ray imaging apparatus 100 includes the X-ray generator 110, the X-ray detector 120, the input unit 130, the controller 140, the display unit 170, the storage unit 180, the table 190, a stereo camera 250, and an image processor 260. The remaining components except for the stereo camera 250 and the image processor 260 have been described above, and accordingly, further descriptions thereof will be omitted.

The X-ray imaging apparatus 100 illustrated in FIG. 10 includes the stereo camera 250, instead of the depth camera 150 of the X-ray imaging apparatus 100 illustrated in FIG. 3.

The stereo camera 250 may photograph an object lying on the table 190 to acquire a stereoscopic image of the object 30. For example, the stereo camera 250 may include a left camera 251 and a right camera 252. The left camera 251 and the right camera 252 are spaced apart from each other by a predetermined distance, where the predetermined distance may be fixed or varied. Each of the left and right cameras 251 and 252 includes an image sensor. The image sensor may be a CCD image sensor, a CMOS image sensor, or another type of image sensor known to those skilled in the art. Since the stereo camera 250 includes two cameras, the stereo camera 250 can acquire two images (that is, left and right images) of the object 30 when photographing the object 30. By combining the left image with the right image, a stereoscopic image of the object 30 can be acquired.

For example, like the depth camera 150, the stereo camera 250 may photograph the object 30 when the object 30 faces the table 190. As another example, the stereo camera 250 may rotate around the object 30 when the gantry 102 rotates. In this case, the stereo camera 250 may acquire left and right images of the object 30 at different positions.

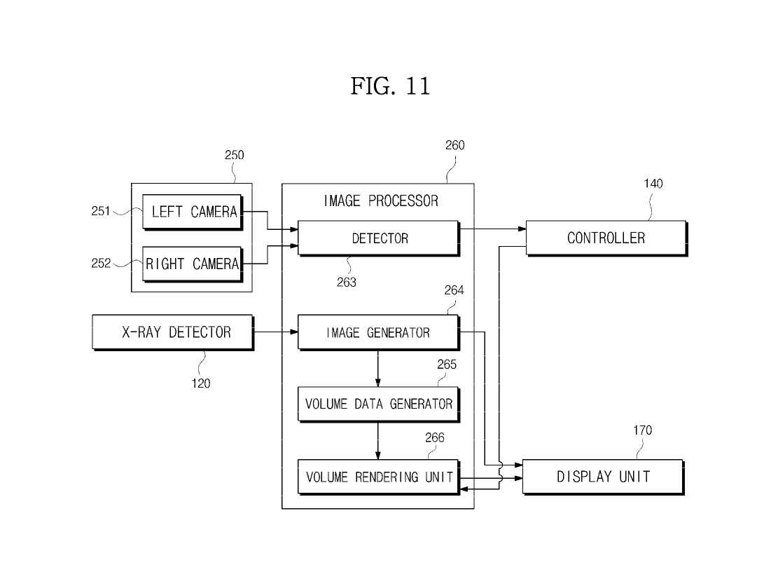

The image processor 260 may acquire position information and thickness information of the object 30 from the left and right images of the object 30. Also, the image processor 260 may generate an X-ray image of the object 30 based on electrical signals output from the individual pixels of the X-ray detector 120. The image processor 260 will be described in more detail with reference to FIG. 11, below.

Referring to FIG. 11, the image processor 260 includes a detector 263, an image generator 264, a volume data generator 265, and a volume rendering unit 266. The image generator 264, the volume data generator 265, and the volume rendering unit 266 may be implemented as the same components as the image generator 164, the volume data generator 165, and the volume rendering unit 166 described above with reference to FIG. 7, and accordingly, further descriptions thereof will be omitted.

The detector 263 acquires position information and thickness information of the object 30 from left and right images of the object 30. When the stereo camera 250 photographs the object 30 while moving around the object 30 so that left and right images of the object 30 are acquired at different positions of the stereo camera 250, the detector 263 acquires position information and thickness information of the object 30 for each position of the stereo camera 250 based on left and right images of the object 30 acquired at the corresponding position of the stereo camera 250. The position information of the object 30 may be used to adjust the position of the table 190, and the thickness information of the object 30 may be used to set a dose of X-rays that is to be irradiated to the object 30.

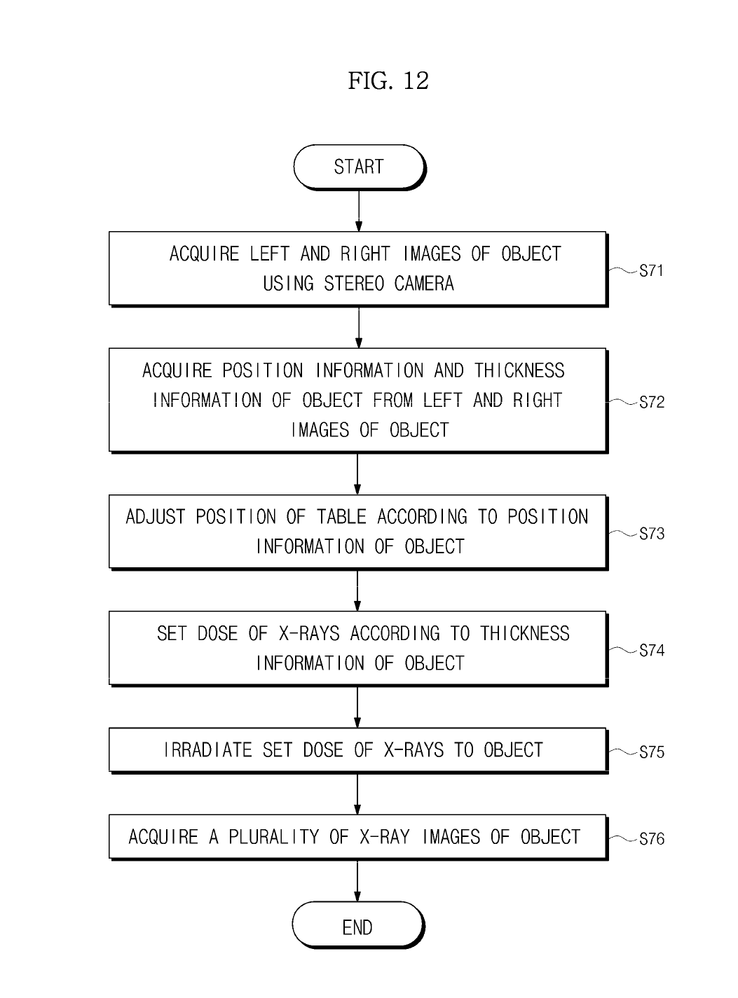

FIG. 12 is a flowchart illustrating a control method of the X-ray imaging apparatus 100 when thickness information of the object 30 is acquired after the stereo camera 250 is fixed.

Referring to FIGS. 1, 10, and 12, first, the table 190 moves to transport the object 30 into the bore 105. Then, the stereo camera 250 moves to face the table 190. In this state, the stereo camera 250 photographs the object 30 to acquire left and right images of the object 30 at operation S71.

Then, position information and thickness information of the object 30 are acquired based on the left and right images of the object 30 at operation S72.

Thereafter, the position of the table 190 is adjusted according to the position information of the object 30 at operation S73. More specifically, a direction in which the table 190 should be moved and a distance by which the table 190 should be moved in order to make a position of the center C_{object} of the object 30 identical to a position of the center C_{bore} of the bore 105 are determined and calculated. Then, the table 190 is moved by the calculated distance in the determined direction.

Then, a dose of X-rays that is to be irradiated to the object 30 is set according to the thickness information of the object 30 at operation S74. For example, a tube voltage may be set in proportion to the thickness of the object 30. As another example, tube current may be set in proportion to the thickness of the object 30. As still another example, it is possible to increase both a tube voltage and tube current in proportion to the thickness of the object 30.

Thereafter, when the gantry 102 rotates to thereby rotate the X-ray generator 110 and the X-ray detector 120 around the object 30, the X-ray generator 110 irradiates the set dose of X-rays to the object 30 at operation S75. At this time, the X-ray generator 110 irradiates the set dose of X-rays to the object 30 regardless of where the X-ray generator 110 is positioned.

By irradiating the set dose of X-rays to the object 30, a plurality of X-ray images of the object 30 can be acquired at operation S76. According to exemplary embodiments, the X-ray image may be one of: a plurality of 2D projected images of the object 30; a 3D image acquired by performing volume rendering on 3D volume data generated based on the plurality of 2D projected images, with respect to a predetermined viewpoint; and a 3D stereoscopic image acquired by performing volume rendering on the 3D volume data with respect to two viewpoints corresponding to different positions to acquire a left image and a right image, and then synthesizing the left image with the right image. The X-ray image may be displayed by the display unit 170 according to a predetermined display method.

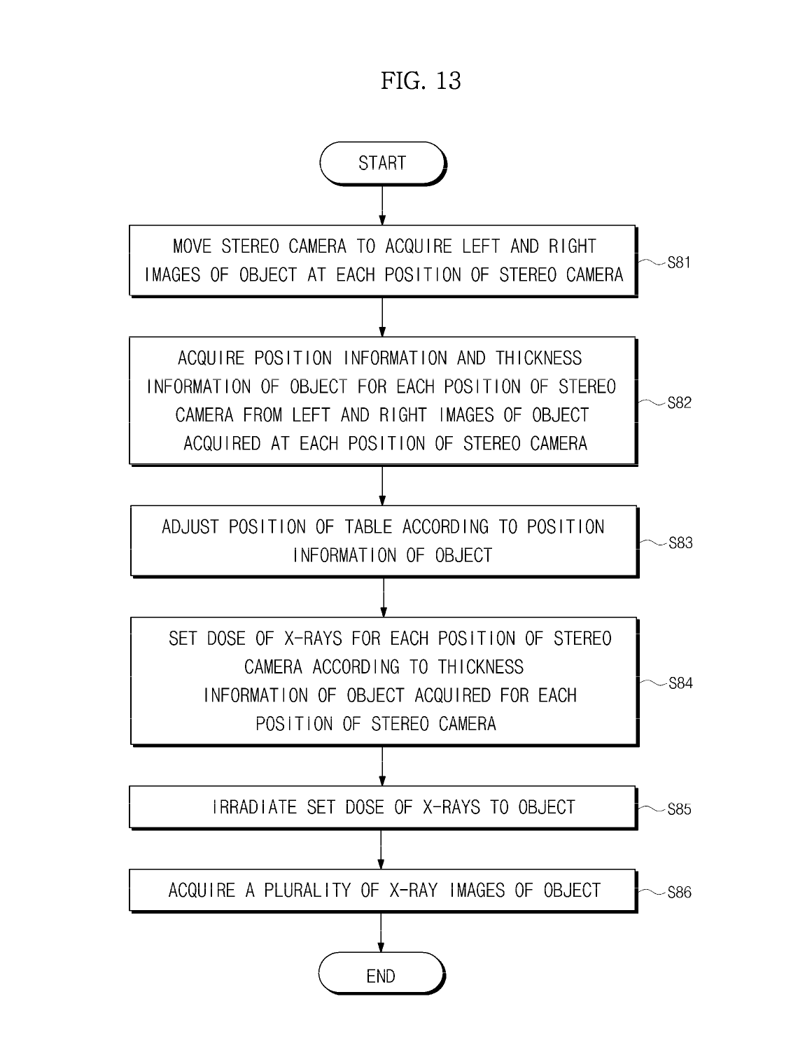

FIG. 13 is a flowchart illustrating a control method of the X-ray imaging apparatus 100 when thickness information of the object 30 is acquired at different positions of the stereo camera 250 moving around the object 30.

Referring to FIGS. 2, 10, and 13, first, the table 190 moves to transport the object 30 into the bore 105. Then, the stereo camera 250 moves to face the table 190. In this state, the stereo camera 250 moves around the object 30 to acquire left and right images of the object 30 at different positions at operation S81. At this time, an operation in which the left camera 251 of the stereo camera 250 photographs the object 30 and an operation in which the right camera 252 of the stereo camera 250 photographs the object 30 may be sequentially performed. The left and right cameras 251 and 252 may read out electrical signals from the individual pixels whenever the stereo camera 250 arrives at one predetermined position among predetermined positions, and acquire left and right images of the object 30 for the corresponding position.

Then, position information and thickness information of the object 30 are acquired for each position of the stereo camera 250 based on the acquired left and right images of the object 30 at operation S82.

Thereafter, the position of the table 190 is adjusted according to the position information of the object 30 at operation S83. More specifically, a direction in which the table 190 should be moved and a distance by which the table 190 should be moved in order to make a position of the center C_{object} of the object 30 identical to a position of the center C_{bore} of the bore 105 are determined and calculated. Then, the table 190 is moved by the calculated distance in the determined direction. At this time, the position information of the object 30 may be selected from among a plurality of pieces of position information of the object 30 acquired at different positions of the stereo camera 250. Alternatively, the position information of the object 30 may be an average value of the acquired pieces of position information.

Then, a dose of X-rays that is to be irradiated to the object 30 for each position is set according to thickness information of the object 30 acquired for the position at operation S84. For example, a tube voltage may be set in proportion to the thickness of the object 30. As another example, tube current may be set in proportion to the thickness of the object 30. As still another example, it is possible to increase both a tube voltage and tube current in proportion to the thickness of the object 30.

Thereafter, when the gantry 102 rotates so that the X-ray generator 110 rotates around the object 30, the X-ray generator 110 irradiates a dose of X-rays set for the corresponding position to the object 30. More specifically, whenever the X-ray generator 110 arrives at a position at which thickness information of the object 30 has been acquired, the X-ray generator 110 irradiates a dose of X-rays corresponding to the position to the object 30 at operation S85.

By irradiating a dose of X-rays corresponding to each position to the object 30, a plurality of X-ray images of the object 30 can be acquired at operation S86. According to exemplary embodiments, the X-ray image may be one of: a plurality of 2D projected images of the object 30; a 3D image acquired by performing volume rendering on 3D volume data generated based on the plurality of 2D projected images, with respect to a predetermined viewpoint; and a 3D stereoscopic image acquired by performing volume rendering on the 3D volume data with respect to two viewpoints corresponding to different positions to acquire a left image and a right image, and then synthesizing the left image with the right image. The X-ray image may be displayed through the display unit 170 according to a predetermined display method.

In the above-described exemplary embodiments, some components constructing the X-ray imaging apparatus 100 may be implemented as modules.

According to exemplary embodiments, the term “module” represents a software element or a hardware element, such as a Field Programmable Gate Array (FPGA) or an Application Specific Integrated Circuit (ASIC), and the module performs a predetermined role. However, the module is not limited to software or hardware. Further, the module may be constructed to exist in an addressable storage module, or to play one or more processors.

For instance, the module includes elements (e.g., software elements, object-oriented software elements, class elements and task elements), processors, functions, properties, procedures, subroutines, segments of a program code, drivers, firmware, a microcode, a circuit, data, a database, data structures, tables, arrays, and variables. Herein, functions provided by components and modules may be provided by a smaller number of combined larger components and modules, or by a larger number of divided smaller components and modules. In addition, the components and modules may be realized to operate one or more CPUs in a device.

In addition to the above-described exemplary embodiments, exemplary embodiments can also be implemented as various types of media (for example, a transitory computer-readable medium) including computer readable codes/commands to control at least one component of the above described exemplary embodiments. The media may be implemented as any medium that can store and/or transmit the computer readable code.

The computer readable code may be recorded on the medium or transmitted through the Internet, and examples of the medium include a magnetic storage medium (e.g., ROMs, floppy disks, hard disks, etc.), an optical recording medium (e.g., CD-ROMs or DVDs), and a transmission medium such as carrier waves. Also, the medium may be a non-transitory computer-readable medium. In addition, the medium may be distributed to computer systems over a network, in which computer-readable code may be stored and executed in a distributed manner. Furthermore, the processing component may include a processor or a computer processor, and may be distributed and/or included in a device.

Although a few exemplary embodiments have been shown and described, it would be appreciated by those skilled in the art that changes may be made in these exemplary embodiments without departing from the principles and spirit of the invention, the scope of which is defined in the claims and their equivalents,

What is claimed is:

1. An X-ray imaging apparatus comprising:
a gantry configured to rotate around an object, the object being placed on a table that is configured to be transported into a bore;
a depth camera provided on the gantry, the depth camera configured to acquire a depth image of the object;
an image processor configured to acquire thickness information of the object from the depth image of the object; and
a controller configured to set a dose of X-rays to be irradiated to the object according to the thickness information of the object.

2. The X-ray imaging apparatus according to claim 1, wherein the image processor is further configured to acquire position information of the object, and the controller is configured to adjust a position of the table based on the position information of the object.

3. The X-ray imaging apparatus according to claim 1, further comprising an X-ray generator configured to irradiate X-rays to the object, and an X-ray detector configured to detect X-rays transmitted through the object,
wherein the depth camera is provided near the X-ray generator.

4. The X-ray imaging apparatus according to claim 1, wherein the depth image of the object is acquired when the depth camera is fixed to face the table.

5. The X-ray imaging apparatus according to claim 1, wherein the controller is configured to set at least one of a tube voltage and a tube current of the X-ray generator in proportion to the thickness information of the object.

6. The X-ray imaging apparatus according to claim 1, wherein the depth image of the object is acquired for a plurality of predetermined positions of the depth camera when the depth camera photographs the object while moving around the object.

7. The X-ray imaging apparatus according to claim 6, wherein the image processor is configured to acquire position information of the object and the thickness information of the object from the depth image of the object acquired for each of the predetermined positions of the depth camera.

8. The X-ray imaging apparatus according to claim 7, wherein the controller is configured to set a dose of X-rays for each of the predetermined positions of the depth camera according to the thickness information of the object acquired for the respective predetermined positions of the depth camera.

9. The X-ray imaging apparatus according to claim 1, wherein the depth camera comprises a structured-light type depth camera or a Time Of Flight (TOF) type depth camera.

10. An X-ray imaging apparatus comprising:
a gantry configured to rotate around an object, the object being placed on a table that is configured to be transported into a bore;
a stereo camera provided on the gantry, the depth camera configured to acquire at least one pair of a left image and a right image of the object;
an image processor configured to acquire thickness information of the object from the at least one pair of the left image and the right image of the object; and
a controller configured to set a dose of X-rays that is to be irradiated to the object according to the thickness information of the object.

11. The X-ray imaging apparatus according to claim 10, wherein the image processor is further configured to acquire position information of the object, and the controller is configured to adjust a position of the table based on the position information of the object.

12. The X-ray imaging apparatus according to claim 10, further comprising an X-ray generator configured to irradiate X-rays to the object, and an X-ray detector configured to detect X-rays transmitted through the object,
wherein the stereo camera is provided near the X-ray generator.

13. The X-ray imaging apparatus according to claim 10, wherein the at least one pair of the left image and the right image of the object is acquired when the depth camera is fixed to face the table.

14. The X-ray imaging apparatus according to claim 10, wherein the controller is configured to set at least one of a tube voltage and tube current of the X-ray generator in proportion to the thickness information of the object.

15. The X-ray imaging apparatus according to claim 10, wherein the at least one pair of the left image and the right image of the object is acquired for a plurality of predetermined positions of the stereo camera when the stereo camera photographs the object while moving around the object.

16. The X-ray imaging apparatus according to claim 15, wherein the image processor is configured to acquire position information of the object and the thickness information of the object from the left image and the right image of the object acquired for each of the predetermined positions of the depth camera.

17. The X-ray imaging apparatus according to claim 16, wherein the controller is configured to set a dose of X-rays for each of the predetermined positions of the stereo camera according to the thickness information of the object acquired for the respective predetermined position of the stereo camera.

18. An imaging method, comprising:
acquiring a depth image of an object;
acquiring thickness information of the object according to the depth image; and
irradiating the object with a quantity of X-rays to thereby acquire an image of the object,
wherein the quantity of the X-rays is determined according to the thickness information.

19. The imaging method according to claim 18, wherein the acquiring of the depth image comprises using a depth camera fixed at a predetermined location.

20. The imaging method according to claim 18, wherein the acquiring of the depth image comprises using a depth camera which rotates around the object and acquires images at a plurality of predetermined points around the object, the depth image being based on the acquired images.

21. The imaging method according to claim 18, further comprising:
acquiring position information of the object, the position information indicating a position of the object relative to a predetermined location in an X-ray imaging apparatus; and
adjusting a location of the object according to the position information.

22. The imaging method according to claim 21, wherein the adjusting of the location comprises adjusting the location such that a center of the object is located in a same position as a center of a bore included in the X-ray imaging apparatus.
